(19) United States
(12) Patent Application Publication
(10) Pub. No.: US 2024/0327797 A1
(43) Pub. Date: Oct. 3, 2024

(54) Fetal Mesenchymal Stem Cells

(71) Applicant: BOOST Pharma ApS, Copenhangen N (DK)

(72) Inventors: Cecilia GÖTHERSTRÖM, Sollentuna (SE); Magnus WESTGREN, Stockholm (SE); Lilian Walther JALLOW, Spånga (SE)

(73) Assignee: BOOST Pharma ApS, Copenhangen N (DK)

(21) Appl. No.: 18/577,414

(22) PCT Filed: Jul. 8, 2022
(86) PCT No.: PCT/EP2022/069097

§ 371 Date: Jan. 8, 2024

(30) Foreign Application Priority Data
Jul. 9, 2021 (EP) 21184760.3

(51) Int. Cl. C12N 5/0775; A61K 35/28; G01N 15/10; G01N 15/149; G01N 33/569

(52) U.S. Cl. CPC C12N 5/0668; A61K 35/28; G01N 15/149; G01N 33/56977; G01N 2015/1006

(57) ABSTRACT

The present invention relates to a population of fetal mesenchymal stem cells (FMSCs). More specifically the present invention relates to FMSCs capable of differentiating into osteoblasts, as well as methods and uses thereof.

### TECHNICAL FIELD

The present invention relates to human fetal mesenchymal stem cells (FMSCs). More specifically it relates to FMSCs capable of differentiating into osteoblasts, as well as methods and uses thereof.

### BACKGROUND

Osteogenesis Imperfecta (01) is a rare metabolic bone disorder that affects ˜1 in 20,000 births. Common features include bone fragility, osteopenia, short stature, atypical skeletal development, brittle teeth, hearing loss and hypermobile joints. Variants in the COL1A1 and COL1A2 genes that either reduce the amount of collagen or impact on collagen structure are causal in 85-90% of cases. OI has been classified as mild (type 1), moderate (type 4), severe (type 3) or lethal (type 2). OI is a heterogeneous condition and severity varies widely, including between family members with the same genetic variant. Severe forms can sometimes be identified before birth when fractures, reduced fetal growth or bowing of the limbs are detected by ultrasound. There are no curative interventions for O. Treatment options focus on reducing fractures and deformity, providing relief from pain and improving mobility and day-to-day function. Bisphosphonates are often offered when children have moderate to severe 01 as they increase bone mineral density and improve bone strength and, as a result, may reduce fracture risk and bone pain. Surgery, such as intramedullary rodding, maintains bony alignment and reduces fracture risks when long bones are bowed or if fractures are recurrent. Physical and occupational therapy are also commonly used to help with mobility and function.

Transplantation of fetal mesenchymal stem cells (FMSCs) is a new approach to treating O that, although it will not cure the condition, does have the potential to modify severity. As OI can cause damage early in fetal life, prenatal or early post-natal (first year of life) stem cell transplantation (SCT) could help to ameliorate the disease process at a time of rapid skeletal development and before additional pathology occurs as a result of fractures. To date clinical experience with SCT for OI is limited, and further studies are needed.

### SUMMARY

The present invention provides a cell culture system for expanding human fetal mesenchymal stem cells (FMSCs) that can differentiate into osteoblasts in vivo.

In one aspect, the present invention provides a population of FMSCs derived from fetal liver, wherein the FMSCs
a) have the ability to expand;
b) are non-tumorigenic;
c) are adherent without growth factors (GF);
d) are CD73+, CD90+, CD45−, CD31−, HLA class II−; and
e) can differentiate into osteoblasts.

In one aspect, the present invention provides a population of FMSCs comprising at least 70% viable cells, such as at least 85% viable cells expressing at least 85% CD73, and CD90, and expressing no more than 5% of CD45, CD31 and HLA class II.

In one aspect, the present invention provides a method for expanding a population of FMSCs comprising culturing said cells in adherent cell culture in culture medium with no growth factors.

In one aspect, the present invention provides a method for enriching a primary fetal liver explant for FMSCs comprising
a) isolation of fetal MSC from the dissected fetal liver tissue;
b) expansion in adherent culture in a culture medium with no growth factors;
c) passaging the cells; and
d) harvesting the cells.

In one aspect, the present invention provides a population of FMSCs as herein described for use in the treatment of osteogenesis imperfecta.

In another aspect the invention relates to a method for release testing of a population of fetal mesenchymal stem cells (FMSCs) derived from fetal liver for use in treatment of osteogenesis imperfecta, said method comprising determining in the population of FMSCs
a) expression of CD73 and CD90; and
b) absence or low expression of CD45, CD31 and HLA class II.

The method of release testing ensures that the population of cells meet certain quality parameters in order to be suitable for use for treatment of osteogenesis imperfecta. This is usually done after a culturing phase, where the cells have been expanded.

The FMSCs described herein have a surprisingly high bone formation capacity which makes them very suitable for treatment of bone defect disorders, such as osteogenesis imperfecta. Once implanted into the recipient, the FMSCs will migrate to the bones and start to differentiate into bone cells, produce healthy collagen and secrete paracrine factors.

### DESCRIPTION OF DRAWINGS

FIG. 1: Flowchart: Manufacturing Process and in-process controls of FMSC cell stock (CS) and drug substance (stages 2-10).

FIG. 2: Morphology of fetal MSC Morphology of FMSC24, Passage 3-4 at magnification 10× at two time-points; Day 2 and Day 6 (before harvest).

FIG. 3: Flow Cytometry plot of data obtained from FMSCs batch Q2/DP4. Upper panels show a staining of the drug product (DP) cells spiked with 4% Peripheral Blood Leucocytes cells (PBL, in red circles) (population in upper right panels, upper left square) as a control that the assay can detect <5% of contaminating cells. Lower panels show DP cells only.

FIG. 4: Gating strategy for phenotyping of the FMSC DP using a spiked control sample consisting of fetal MSC with 4% of PBMC.

FIG. 5: Bone differentiation of FMSC drug product. Data from one representative bone differentiation experiment performed over 17 days. Tested FMSCs are Q8/DP1 (upper panel) and Control cells (FMSC26), (lower panel). The figure shows the full well of a 12-well plate.

### DETAILED DESCRIPTION

### Definitions

As used herein the term “mesenchymal stem cell” is intended to mean a cell that gives rise to a cell of mesenchymal lineage.

The term “expanded” is herein intended to mean that the resultant cell population is derived from an ex vivo culture of cells cultured in the presence of additives, and where the number of cultured cells exceed the number of non-cultured cells put into said culture at the starting point of the culture, i.e. before expansion.

The term “differentiated” is herein intended to mean that a resultant a cell is committed to a restricted development. An MSC differentiating to an osteoblast is thus a cell that is committed to an osteoblast lineage. Accordingly, “differentiation” is the process by which an unspecialized (“uncommitted”) or less specialized cell acquires the features of a specialized cell. A differentiated or differentiation induced cell is one that has taken on a more specialized (“committed”) position within the lineage of a cell.

As used herein the term “cell-culture” is intended to mean the maintenance of cells in an artificial in vitro environment. It is to be understood that the term “cell culture” is a generic term and may be used to encompass the cultivation not only of individual cells but also of tissues, and organ systems.

The term “isolated” as used herein refers to a cell, cellular component, or a molecule that has been removed from its native environment.

The term “osteoblast” is herein intended to mean a differentiated cell that can synthesize bone. Similarly, a cell with an osteoblast phenotype shows most or all characteristics of an osteoblast.

As used herein, the term “non-human animal products” refer to serum, plasma, growth factors and other cell culturing reagents that are used for the culturing and growth of the FMSCs, that are derived from non-humans. For example, fetal calf serum, fetal bovine serum, mouse basic fibroblast growth factor and recombinant mouse basic fibroblast growth factor are considered non-human animal products.

As used herein, the term “non-tumorigenic” refer to a non-tumor forming property.

As used herein, the term “adherent cell” refers to a cell which must be attached to a surface to grow.

### Fetal Mesenchymal Stem Cells

The invention relates to methods for culture-expansion of human fetal mesenchymal stem cells (FMSCs), and the cryopreservation of FMSCs to yield a composition of expanded fetal derived mesenchymal stem cells. FMSCs can be stored frozen, subsequently thawed and used for differentiation into osteoblast.

In one aspect, the present invention provides a population of FMSCs derived from fetal liver, wherein the FMSCs
a) have the ability to expand;
b) are non-tumorigenic;
c) are adherent without GF;
d) are CD73+, CD90+, CD45−, CD31−, HLA class II−; and
e) can differentiate into osteoblasts.

In one embodiment, the fetal liver is isolated from elective abortion in the first trimester of the pregnancy. The FMSCs are derived from first trimester liver tissue from elective surgical (vacuum aspiration) terminations.

In one embodiment, the FMSCs are highly bone forming.

In one embodiment, the differentiation of the FMSCs into bone is at least two fold differentiation over negative control, such as 2.5 fold differentiation over negative control, such as 3 fold differentiation over negative control, such as 3.5 fold differentiation over negative control, such as 4 fold differentiation over negative control, such as 4.5 fold differentiation over negative control, such as 5 fold differentiation over negative control, such as 5.5 fold differentiation over negative control, such as 6 fold differentiation over negative control, such as 6.5 fold differentiation over negative control, such as 7 fold differentiation over negative control, such as 7.5 fold differentiation over negative control, such as 8 fold differentiation over negative control, such as 8.5 fold differentiation over negative control, such as 9 fold differentiation over negative control, such as 9.5 fold differentiation over negative control, such as 10 fold differentiation over negative control, such as 15 fold differentiation over negative control, such as 20 fold differentiation over negative control, such as 25 fold differentiation over negative control, such as 30 fold differentiation over negative control, such as 35 fold differentiation over negative control, such as 40 fold differentiation over negative control, such as 45 fold differentiation over negative control, such as 50 fold differentiation over negative control, such as 55 fold differentiation over negative control.

In one embodiment, the FMSCs can expand and grow for 13 passages.

In one embodiment, the FMSCs have a Hayflick limit.

In one embodiment, the population of FMSCs is at least 85% CD73+. In one embodiment, the population of FMSCs is at least 80% CD73+, such as at least 85% CD73+, for example at least 90% CD73+, such as at least 95% CD73+, such as at least 98% CD73+.

Preferably, the population of FMSCs is at least 85% CD73+.

In one embodiment, the population of FMSCs is at least 85% CD90+. In one embodiment, the population of FMSCs is at least 80% CD90+, such as at least 85% CD90+, for example at least 90% CD90+, such as at least 95% CD90+, such as at least 98% CD90+.

Preferably, the population of FMSCs is at least 85% CD90+.

In one embodiment, the population of FMSCs is at least 70% live cells. In one embodiment, the population of FMSCs is at least 80% live cells, such as at least 85% are live cells, for example at least 90% are live cells, such as at least 95% live cells, such as at least 98% are live cells.

Preferably, the population of FMSCs is at least 70% are live cells.

In one embodiment, the population of FMSCs is no more than 5% CD45+, for example no more than 4% CD45+, such as nor more than 3% CD45+, for example no more than 1% CD45+.

Preferably, the population of FMSCs is no more than 5% CD45+.

In one embodiment, the population of FMSCs is no more than 5% CD31+, for example no more than 4% CD31+, such as nor more than 3% CD31+, for example no more than 1% CD31+.

Preferably, the population of FMSCs is no more than 5% CD31+.

In one embodiment, the population of FMSCs is no more than 5% HLA class II+, for example no more than 4% HLA class II+, such as nor more than 3% HLA class II+, for example no more than 1% HLA class II+.

Preferably, the population of FMSCs is no more than 5% HLA class II+.

In one aspect, the present invention provides a population of FMSCs having the ability to differentiate into osteoblasts. The population of FMSCs may differentiate into osteoblasts in vitro and in vivo.

In one aspect, the present invention provides a population of FMSCs comprising at least 70% viable cells, such as at least 85% viable cells expressing, at least 85% CD73, and CD90, and expressing no more than 5% of CD45, CD31 and HLA class II, respectively.

Preferably, the population of FMSCs comprise at least 90% viable cells expressing, at least 90% CD73, and CD90, and expressing no more than 5% of CD45, CD31 and HLA class II.

In one aspect, the present invention also provides a method for release testing of a population of fetal mesenchymal stem cells (FMSCs) derived from fetal liver for use in treatment of osteogenesis imperfecta, said method comprising determining in the population of FMSCs
a) expression of CD73 and CD90; and
b) absence or low expression of CD45, CD31 and HLA class II.

In one embodiment, according to the method for release testing, the population of FMSCs is determined to have at least 70% viable cells expressing at least 85% CD73, and at least 85% CD90, and no more than 5% of CD45, CD31 and HLA class II.

The methods of determining expression of cell surface markers would be known to the person skilled in the art. For example, the expression of the markers can be confirmed using common immunophenotyping techniques, where antibody is used to identify cells based on the types of antigens or markers expressed, such as flow cytometry or immunocytochemistry.

Accordingly, in one embodiment the release testing comprises immunophenotyping.

In one embodiment, the immunophenotyping is done by flow cytometry.

In one embodiment, the immunophenotyping is done by immunocytochemistry.

In one embodiment, the methods for release testing further comprises determining the population of FMSCs that is able to expand, is non-tumerigenic, and is able to differentiate into osteoblasts.

In one embodiment, the methods for release testing further comprise characterizing the population as being one or more of sterile, virus free, mycoplasma free, exhibiting chromosome stability, being free of known mutations causing skeletal dysplasia, and being a colourless cell suspension free of visible particulate matter.

### Methods for Expanding

In one aspect, the present invention provides a method for expanding a population of FMSCs comprising culturing said cells in adherent cell culture in culture medium with no growth factors.

In one embodiment, the culture medium is DMEM.

### Methods for Enriching

In one aspect, the present invention provides a method for enriching a primary fetal liver explant for FMSCs comprising
a) isolation of fetal MSC from the dissected fetal liver tissue;
b) expansion in adherent culture in a culture medium without growth factors;
c) passaging the cells; and
d) harvesting the cells.

In one embodiment, passaging the cells comprises removing dead cells and/or non-MSC by washing such that only adherent cells remain in the culture.

In one embodiment, the cells are passaged at 70-100% confluency.

### Osteogenesis Imperfecta

In one aspect, the present invention provides a population of FMSCs as described herein for use in the treatment of osteogenesis imperfecta.

In one embodiment, the population of FMSCs may be used for improving the symptoms of osteogenesis imperfecta.

Treating,” or “Treatment,” as used herein, includes any administration or application of a therapeutic for the disclosed diseases, disorders and conditions in subject, and includes inhibiting the progression of the disease, slowing the disease or its progression, arresting its development, partially or fully relieving the disease, or partially or fully relieving one or more symptoms of a disease.

In one embodiment, the population of FMSCs is suitable for allogenic transplantation.

In one embodiment, the population of FMSCs may be mismatched with the recipient. The mismatch may be a genetic mismatch, a sex mismatch and/or an HLA class mismatch. In one embodiment, the population of FMSCs may be detected in the recipient.

In one embodiment, the population of FMSCs is administered intravenously.

In one embodiment, the population of FMSCs is administered intraosseously.

Intraosseous injections may be performed as described by Ramesh et al., 2021.

In one embodiment, the population of FMSCs is administered postnatally and/or prenatally.

In one embodiment, the population of FMSCs is administered four times postnatally.

In one embodiment, the population of FMSCs is administered one time prenatally and three times postnatally.

In one embodiment, the population of FMSCs is administered every 3 months. In one embodiment, the population of FMSCs is administered every 4 months. In one embodiment, the population of FMSCs is administered every 5 months. In one embodiment, the population of FMSCs is administered every 6 months. In one embodiment, the population of FMSCs is administered every year.

In one embodiment, the population of FMSCs may be administered at any time points. For example, the population of FMSCs may be administered upon patient's worsening of symptoms and/or decreased bone density.

In one embodiment, the population of FMSCs is administered in the amount of 0.5×10⁶ viable FMSCs/kg body weight, such in the amount of 1×10⁶ viable FMSCs/kg body weight, such in the amount of 1.5×10⁶ viable FMSCs/kg body weight, such in the amount of 2×10⁶ viable FMSCs/kg body weight, such in the amount of 2.5×10⁶ viable FMSCs/kg body weight, such in the amount of 3×10⁶ viable FMSCs/kg body weight, such in the amount of 3.5×10⁶ viable FMSCs/kg body weight, such in the amount of 4×10⁶ viable FMSCs/kg body weight, such in the amount of 4.5×10⁶ viable FMSCs/kg body weight, such in the amount of 5×10⁶ viable FMSCs/kg body weight, such in the amount of 5.5×10⁶ viable FMSCs/kg body weight.

In one embodiment, the present invention provides a composition comprising the population of FMSCs as described herein and a pharmaceutically acceptable carrier.

In one embodiment, the composition comprises FMSCs and human serum albumin (HSA) and physiological saline. In a preferred embodiment, the composition comprises 2×10⁶/ml viable FMSCs, 8% (v/v) Human serum albumin (HSA) and 92% (v/v) physiological saline.

### EXAMPLES

### Example 1—Manufacturing Process for FMSC Product

The manufacturing of FMSC product starts with the isolation of FMSCs from the dissected fetal liver tissue from the procured fetal tissue. The fetal MSC are prepared as a cell suspension and expanded in adherent culture using cell culture flasks. The process is started by expansion into one FMSC cell stock and subsequently to several FMSC drug product batches; each MSC drug product batch is started from one cryopreserved FMSC cell stock vial (2.5×10⁶ cells-3×10⁶ cells). A FMSC cell stock consists of 10×10⁶-45×10⁶ viable cells depending on the size of the fetal liver. The batch size of each FMSC drug product batch is approximately 250×10⁶-450×10⁶ viable cells. The cell viability is determined by live/dead exclusion staining.

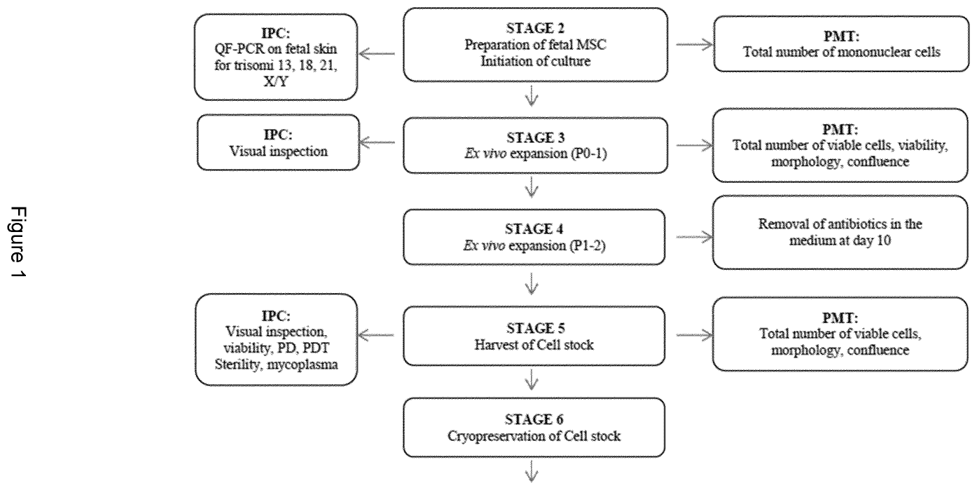

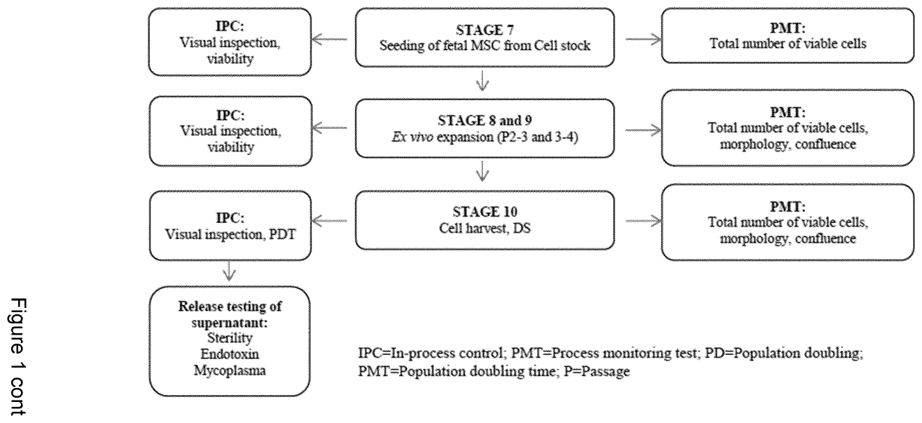

A flowchart of the manufacturing process from the isolation of fetal MSC, expansion and collection of cells for the FMSC cell stock, re-initiation of culture from the FMSC cell stock and further expansion to the cell harvest step of the sub-batches of FMSC drug substance (DS) with the in-process controls performed during the manufacturing process is shown in FIG. 1.

### Summary of Manufacturing and in Process Controls of the FMSC Drug Substance

The fetal liver tissue is prepared as a cell suspension and expanded in adherent culture using plastic cell culture flasks with Dulbecco's Modified Eagle Medium (DMEM) with 10% V/V fetal bovine serum (FBS). The batch size of the FMSC cell stock is approximately 15-45×10⁶ viable MSC (two passages) and takes approximately 2 to 3 weeks to prepare. The manufactured FMSC cell stock is aliquoted and cryopreserved in 10% V/V Dimethyl sulfoxide (DMSO) and 10% V/V FBS in DMEM using a temperature controlled cryo preservation method and stored at <−150° C. in a freezer in vials containing 2.5-3.0×10⁶ cells/vial. Analysis of sterility and mycoplasma is performed as in-process control (IPC) on the FMSC cell stock. From one FMSC cell stock vial one FMSC drug product is manufactured. Each FMSC drug product batch consists of 250-450×10⁶ viable FMSC. The manufacturing time per FMSC drug substance is approximately 2 weeks. The cumulative population doublings (PD) and the population doubling time (PDT) are followed throughout the manufacture. PD is a release test for the FMSC drug product (maximum 13 PD in total from passage 1). PDT is an in-process control for the FMSC cell stock (35-300 h) and a release criterion for the FMSC drug substance (35-65 h). Throughout the expansion, Process monitoring tests (PMT) are performed (cell count, viability count, control of morphology and cell confluency and inspection of cell culture medium to detect signs of contamination). In-process control include viability (>80%) and PD and PDT as described above. With every passage, dead cells and non-MSCs are removed by washing and only adherent cells remain in the culture. The cells are passaged at 70-100% confluence as determined by visual inspection, dissociation with TrypLETM Select, and one subsequent washing and centrifugation step. The FMSC drug substance is defined as the cell pellet after dissociation from plastic surface with TrypLETM Select (prior to resuspension in cryomedium) and hence this is the last step of the manufacturing of the FMSC drug substance.

The release testing for sterility, mycoplasma and endotoxins are performed on the FMSC drug substance while cell phenotype and potency is conducted on the FMSC drug product. In addition mycoplasma testing is also performed on the FMSC drug product.

### Stage 1: Procurement of Starting Material

Isolation of the starting material (fetal liver) from the source (fetus) is performed by vacuum aspiration abortion performed. After removal of the cannula the fetal tissues is transferred to a sterile container sealed with a screw cap, with 250 mL sterile 0.9 mg/mL NaCl and 0.25 mL Heparin 5000 IE/mL.

### Stage 2: Initiation of Culture

At the manufacturing site the tissue is placed in a 70 μm cell strainer, mounted on a 50 mL Falcon tube and washed with 2×15 mL of PBS before passaging the tissue through the cell strainer by using a piston from a 1 mL syringe. The cell strainer is washed with room tempered Complete medium (CM) with antibiotic and antimycotic (CM A/A). CM A/A contains DMEM, 10% FBS (V/V), Gensumycin (40 μg/mL, 0.1% V/V) and Fungizone (0.25 μg/mL, 0.1% V/V). Gensumycin and Fungizone are added during manufacture up to day 10. The cells are resuspended and the number of cells is determined by manual counting using a haemocytometer (BQrker chamber) using Turk solution. Türk solution destroys red blood cells and stains mononuclear cells. The total number of mononuclear cells is determined by counting all stained cells; the number of obtained cells depends on the fetal age of the source. Cells are seeded (P0) on a flat (2D) polystyrene surface in culture flasks (25, 75 or 150 cm²) with filter caps in CM A/A at a cell density of 75,000-150,000 cells/cm² and incubated in a humidified 37° C. incubator with 95% (V/V) air, 5% (V/V) CO2. Process monitoring tests for Stage 2 is total number of mononuclear cells and in-process control is QF-PCR on fetal skin to detect trisomy 13, 18, 21 and X/Y.

### Stage 3: Ex Vivo Expansion (P0-1)

24-72 h after initiation of culture all CM A/A is removed, an equal volume of fresh CM A/A is added and the incubation is allowed to continue. CM A/A is aspirated off and replaced with an equal volume of fresh CM A/A every 3-4 days. Up to three medium exchanges could be needed before passaging.

After 7-10 days and before passaging of the cells, the following process monitoring tests are performed: Visual inspection of the flasks for contamination, cell confluency and cell morphology. Upon visual inspection for contamination, the culture medium needs to be clear. Culture flasks containing medium that is not clear are removed from culture and tested for microbial contamination using a sterility test according to Ph. Eur. 2.6.27, and the whole batch is discarded. When the cell culture reaches a confluency of 70-100% the cells in culture are passaged.

Passaging of cells is done by aspirating all medium from the cell culture flasks. Cells are washed once with PBS before the addition of TrypLETM Select (1.5/3/5 mL per 25/75/150 cm² flask) in order to detach the cells from the surface of the flasks. Cells are incubated at 37C° for 3-5 minutes and are followed by visual inspection until the cells are detached. CM A/A is added to the cell culture flasks to inactivate the TrypLETM Select. The suspension containing the cells is collected by aspiration, centrifuged in 50 mL tubes (500×g, 7 minutes) and re-suspended in CM A/A. The cells are counted by Eosin exclusion and are re-plated at a cell density of 3000-5000 cells/cm² in new cell culture (75/150/300 cm²) flasks and incubated as described in Stage 2. Process monitoring tests for Stage 3 are total number of viable cells, viability, morphology and confluence. In-process control is visual inspection.

### Stage 4: Ex Vivo Expansion (P1-2)

Cells are cultured and medium exchanged as described in Stage 3 above. At this passage the antibiotic and anti-mycotic is removed from the cell culture medium at day 10. After 7-10 days, when the culture has reached a confluency of 70-100% the cells are ready for harvest as described in Stage 5.

### Stage 5: Harvest of Fmsc Cell Stock

The cell medium is collected for the following in-process control: Sterility testing and mycoplasma. 2×1 mL aliquots are collected in 2 mL cryovials and are frozen at −80° C. for mycoplasma qPCR. An aliquot of 5 mL is frozen at −80° C. as a retention sample and the remaining volume of culture medium is sent for sterility testing. After removal of the medium, the cells are detached from the culture flasks, washed and counted as described in Stage 3 and the following in-process control is performed: Visual inspection, viability, population doubling and population doubling time, sterility and mycoplasma. Process monitoring tests for Stage 5 are total number of viable cells, morphology and confluence.

### Stage 6: Cryopreservation of Fmsc Cell Stock

At this stage of the expansion process the FMSC cell stock collected in Stage 5 is cryopreserved. The concentration of viable cells is calculated, and the cell suspension is diluted in an appropriate volume to 5-6×10⁶ viable cells/mL of CM. 0.5 mL cell suspension is transferred to 2 mL cryovials and 0.5 mL pre-cooled cryo medium-FBS (20% (V/V) DMSO/10% (V/V) FBS in DMEM) is added. Each vial contains 2.5-3×10⁶ viable cells in 1 mL cryo medium-FBS. The vials are labelled and transferred to a temperature-controlled cryo vessel and subsequently cryopreserved first at −80° C. and within 24-72 h transferred to an ultra-low freezer at ≤−150° C.

Stage 7: Seeding of Fetal MSC from Cell Stock

For the manufacture of a FMSC drug product, one cryovial of the cryopreserved FMSC cell stock is thawed by placing the 2 mL cryovial between the palms of the operator's hands. The 5 mL vial is thawed in a water bath. One mL of CM is added to the cryovial, the cell suspension is mixed once and transferred to a 50 mL tube containing CM and thereafter centrifuged for 7 minutes at 500×g at RT. After centrifugation, the cell pellet is re-suspended in CM and counted as described in Stage 3. After establishing the total viable cell number, the cells are seeded at a cell density of 3000-5000 viable cells/cm² on a flat (2D) polystyrene surface in 300 cm² cell culture flasks in CM. Process monitoring tests for Stage 7 is total number of viable cells and in-process control is visual inspection and viability.

### Stage 8 and Stage 9: Ex Vivo Expansion (P2-3) and (P3-4)

This ex vivo expansion step is the same as Stage 3. Culture medium (without A/A) is refreshed every 3-4 days by removing the medium from the culture flask and replacing it with fresh culture medium. When 70-100% confluency is reached, the cells are passaged as described in Stage 3. Passaging is performed to allow the cells to grow and expand optimally. At the end of Stage 9 the cells have been passaged four times, twice from culture initiation to FMSC cell stock (P0-1 and P1-2) and twice from FMSC cell stock to final harvest (P2-3 and P3-4). Process monitoring tests for Stages 8 and 9 are total number of viable cells, morphology and confluence. In-process control are visual inspection and viability.

### Stage 10: Cell Harvest, FMSC Drug Substance

After the last cell passaging, cells are collected and centrifuged. After centrifugation, the cell pellet is re-suspended in CM and counted as described in Stage 3. The cells are centrifuged a second time and the supernatant is removed. The resulting pellet is the FMSC drug substance. Process monitoring tests for Stage 10 are total cell number, morphology and confluence. In-process control are visual inspection and population doubling time. Release testing performed is sterility, endotoxin and mycoplasma on supernatant before harvest.

### Example 2—Cell Characteristics and Cell Expansion

### Cell Morphology

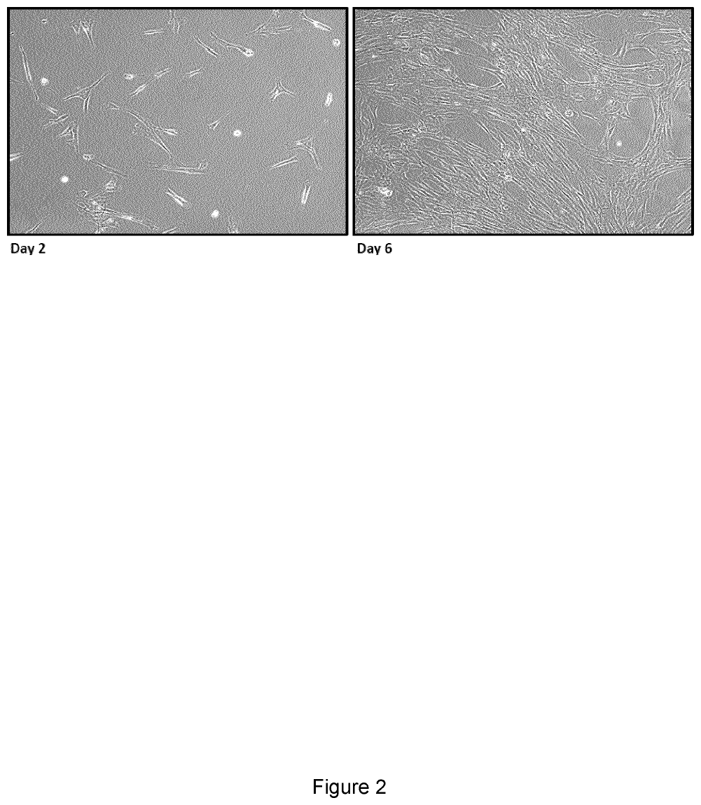

The morphology of cells is evaluated by microscopy during ex vivo expansion by visual inspection of the cells in the culture flask at 10× magnification. FIG. 2 shows an image of the morphology of a typical fetal MSC culture from ex vivo expansion. Cells are spindle shaped and attach to the tissue culture flask. When the cell culture reaches a confluency of 70-100% the cells are passaged.

### Cell Expansion

The cell expansion from the starting material to the FMSC cell stock was followed over time. The data are presented in Table 1. FMSC cell stock could be manufactured from all starting material. The starting material (three fetal livers, Q2, Q4, and Q8) was of similar fetal age, but the number of total nucleated cells in the starting material varied, which influenced the amount of cells obtained after manufacture. The data also show that the confluency at harvest impacts the total number of cells harvested.

TABLE 1
Summary of the manufacture of three FMSC cell
stocks (CS) to five FMSC drug products (DP)

| | | Q2 | | | Q4 | Q8 |
|---|---|---|---|---|---|---|
| | Characteristics | DP2 | DP3 | DP4 | DP1 | DP1 |
| P2- | Number of viable | 4.6 | 3.3 | 3.4 | 0.9 | 2.0 |
| 3 | cells (×10⁶) at | | | | | |
| | thawing of CS | | | | | |
| | Cultivation area | 900 | 900 | 900 | 300 | 600 |
| | (cm²) | | | | | |
| | Confluency (%) | 100 | 100 | 100 | 90 | 100 |
| | Population | 2.8 | 3.9 | 3.8 | 3.6 | 4.1 |
| | doublings | | | | | |
| | Population | 59.7 | 42.9 | 43.4 | 46.2 | 40.4 |
| | doubling time | | | | | |
| | Number of viable | 31.7 | 49.0 | 48.3 | 11.0 | 34.2 |
| | cells (×10⁶) | | | | | |
| P3- | Cultivation area | 9000 | 9000 | 9000 | 3600 | 7800 |
| 4 | (cm²) | | | | | |
| | Confluency (%) | 95 | 100 | 90 | 70* | 95 |
| | Population | 3.6 | 3.4 | 3.3 | 2.7 | 3.4 |
| | doublings | | | | | |
| | Population | 45.4 | 42.7 | 43.3 | 62.4 | 42.3 |
| | doubling time | | | | | |
| | Total number of | 381.8 | 509 | 487.2 | 70.9 | 357.2 |
| | viable cells | | | | | |
| | (×10⁶) | | | | | |
| | in DS | | | | | |
| DP | Total number of | 10.1 | 10.9 | 10.9 | 7.5 | 11.7 |
| | population | | | | | |
| | doublings from | | | | | |
| | P1 | | | | | |
| | Number of DP | 89/1 | 64/12 | 59/12 | 16/0 | 52/5 |
| | vials (1 mL/3 | | | | | |
| | mL) | | | | | |
| | Number of viable | 5 | 5 | 5 | 5 | 5 |
| | cells/DP vial | | | | | |
| | (×10⁶) at | | | | | |
| | cryopreservation | | | | | |

### Expansion of Fetal MSC Beyond Final Manufacturing at Passage 3-4

During manufacture of the drug product, the FMSC drug product is harvested at P3-4. The maximum allowed number of PD from P1 is 13. To verify that the cells can be expanded further without transformation and that the cells are functional beyond the expansion in this manufacturing process, three batches of FMSC drug product were expanded for two additional passages (P4-5 and P5-6) using the same manufacturing method as for the FMSC drug product. Data from harvest at P3-4 (DP) was compared to data from P5-6 regarding bone differentiation, phenotype and karyotype. The cells retained their phenotype, their capacity to differentiate to bone and showed no abbreviations with additional expansion and passaging as shown by karyotyping. In one FMSC drug product, 3 ring chromosomes were detected in 26 metaphases analysed at P3-4. At P5-6 the ring chromosomes were not present any longer which, according to information from the Clinical genetics lab, performing the karyotyping, can sometimes be seen during cell culture. The ring chromosomes are derived from the donor and are not acquired during culture expansion.

TABLE 2
Comparison of results from P3-4 to P5-6

| | Q2/DP4 | | Q4/DP1 | | Q8/DP1 | |
|---|---|---|---|---|---|---|
| Characteristics | P3-4 | P5-6 | P3-4 | P5-6 | P3-4 | P5-6 |
| Total | 10.9 | 19.8 | 7.5 | 14.9 | 11.7 | 18.4 |
| number of | | | | | | |
| PD from P1 | | | | | | |
| CD73 (%) | 99.5 | 100 | 99.9 | 99.1 | 99.9 | 99.8 |
| CD90 (%) | 99.4 | 99.6 | 99.4 | 99.8 | 97.8 | 96.3 |
| CD31 (%) | 0.7 | 0.9 | 2.4 | 1.0 | 0.7 | 0.7 |
| CD45 (%) | 0.3 | 0.4 | 0.7 | 0.3 | 0.4 | 0.2 |
| HLA class II | 0.4 | 0.3 | 1.1 | 0.6 | 0.4 | 0.3 |
| (%) | | | | | | |
| Bone | 6.3 | 4.2 | 7.2 | ND* | 6.6 | 7.6 |
| differentiation | | | | | | |
| (fold | | | | | | |
| differentiation) | | | | | | |
| Karyotyping | 47, XX, +mar(3)/ | 46, XX | 46, XX | 46, XX | 46, XX | 46, XX |
| | 46, XX(23)** | | | | | |

*Not done due to technical reasons;
**Ring chromosome detected.

### Hayflick Limit Testing of Fetal MSC Cultures

Normal, non-immortalized human somatic fetal cells stop dividing before they reach the Hayflick limit (40-60 population doublings). At this point they enter a senescent phase, which can be indicatively measured by positive cell staining for Beta-Galactosidase. Three FMSC drug product batches were thawed, and the cells were grown and passaged as described for manufacturing of FMSC drug product. The cells were kept in culture beyond the point where they stopped dividing. As expected, the population doubling time increased with increasing passages while the PD decreased with increasing passages.

Expression of Markers Associated with Pluripotency

Expression of markers associated with pluripotency and stem cells by fetal MSC was investigated. Expression of Nanog and Oct-4A/B was analysed using qPCR, and Tra-1-60, Tra-1-81 and SSEA-4 was analysed with flowcytometry. Fetal MSC from 11 donors were tested. None of the samples expressed Nanog, Oct-4A/B, Tra-1-60 or Tra-1-81 but all samples expressed SSEA-4. Expression of SSEA-4 is not coupled to pluripotency and MSC express SSEA-4.

### Example 3—Composition of the Drug Product

The composition of FMSC drug product is:

FMSC drug product—a cell pellet after dissociation from plastic surface at final harvest.

The cell pellet consists of expanded fetal-derived MSC.

Excipients—physiological NaCl (80%, V/V), HSA (10%, V/V) and DMSO (10%, V/V).

The components of FMSC drug product are presented in Table 3.

TABLE 3
Composition of FMSC drug product

| Component | Function | Amount per mL* |
|---|---|---|
| Fetal MSC (FMSC drug | Active substance | 5 × 10⁶ viable cells** |
| substance) | | |
| HSA | Cryoprotectant and cell stabiliser | 10% (V/V) |
| Physiological saline (0.9% NaCl) | Solvent, isotonic agent | 80% (V/V) |
| DMSO | Cryoprotectant | 10% (V/V) |
| Cryovial with external screw cap | Sterile primary container made | 2 or 5 mL cryovial containing 1 |
| without gasket | of high-density polypropylene | or 3 mL DP, respectively |

*dose is 3 × 10⁶ cells/kg bodyweight

The components of the reconstituted FMSC drug product are presented in Table 4.

TABLE 4
Components of the reconstituted FMSC drug product

| Component | Function | Amount |
|---|---|---|
| Fetal MSC (FMSC drug product) | Active single cell population | 2 × 10⁶ viable cells/mL |
| HSA | Solvent and cell stabiliser | 8% (V/V) of excipient volume |
| Physiological saline (9 mg/mL | Solvent, isotonicity agent | 92% (V/V) of excipient volume |
| NaCl) | | |

### Example 4—Control of Drug Product

### Specification(s)

The specifications for release testing of the FMSC drug product comprises of tests on samples which are taken in the manufacturing process before the final FMSC drug product, and on the final FMSC drug product. The release testing, methods and corresponding acceptance criteria are listed in Table 5, indicating the stage of the manufacturing process being tested in each case.

TABLE 5
Specifications for the Control of FMSC drug product 1

| Test | Method | Acceptance criteria | Comments |
|---|---|---|---|
| Population doublings | Cell count | ≤13 cell doublings from | Performed on DP after |
| | | passage 1 | thawing |
| Viability (at thawing) | Cell count | ≥70% | |
| Recovery (at thawing) | Cell count | ≥3.5 × 10⁶ viable | |
| | | cells/mL | |
| % viable fetal-derived | Flow cytometry | ≥85% for | |
| MSC | analysis | CD73& CD90 | |
| % contaminating cells | | ≤5% for CD31 | |
| | | ≤5% for CD45 | |
| | | ≤5% for HLA class II | |
| Bone differentiation | Bone differentiation | ≥2.5-fold differentiation | |
| | assay (in vitro) | over negative control | |
| Chromosome stability | Karyotyping | 46, XX or 46, XY2 | |
| Skeletal dysplasia | DNA sequencing | Negative for known | |
| sequencing2 | | mutations causing | |
| | | skeletal dysplasia | |
| Virus screening3 | PCR | Negative for Zika virus | |
| Virus screening4 | PCR | Negative for Zika, CMV | |
| | | and Parvo virus | |
| Sterility | Ph. Eur. 2.6.1 | Sterile | Performed on DP4 after |
| Mycoplasma | Ph. Eur. 2.6.14 | No mycoplasma | thawing (cells) and on |
| | | detected | DS before freezing |
| | | | (supernatant)¹ |
| Endotoxin | Ph. Eur. 2.6.7 (qPCR) | ≤0.5 EU/mL | On DS before freezing |
| | | | (supernatant)¹ |
| Appearance4 | Visual inspection | Colourless cell | On DP before freezing |
| | | suspension, free of | (cell suspension) |
| | | visible particulate | |
| | | matter | |

¹Sterility, mycoplasma and endotoxin data are further described in the S-section.
2≥2 identical clonal numerical abnormal metaphases on 20-25 metaphases is not accepted.
3Performed on one DP batch per donor
4Testing on the DP will be performed on future batches.

### Analytical Procedures

### Population Doublings and Population Doubling Time

Population doubling time (PDT) and population doubling (PD) are calculated based on the results of the cell counting. PDT is the amount of time it takes for a cell population to double in number at an exponential growth rate. PD is the number of doublings the cell culture has undergone during the time between passages.

Both parameters can be calculated for each passage using the following formula:

PDT=t*1n2

In(N1/NO)

There are set criteria for both parameters and any deviations (between FMSC drug products and/or within one FMSC drug product) are monitored and evaluated. Trend analysis for each FMSC drug product is performed.

The total number of PD from P1 to harvest of FMSC drug substance must not exceed 13.

Determination of the percentage of fetal MSC (CD73+CD90+) The identity and purity of the FMSC drug product were analysed by flow cytometry.

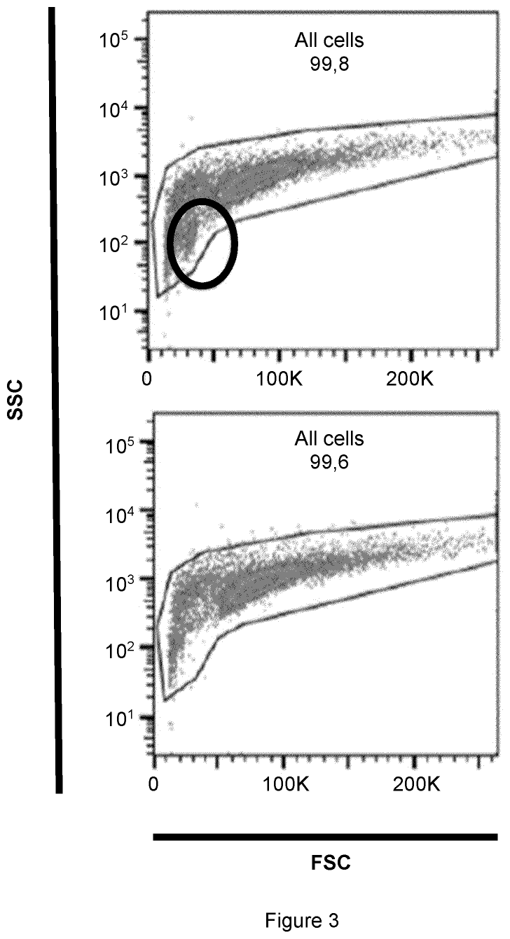

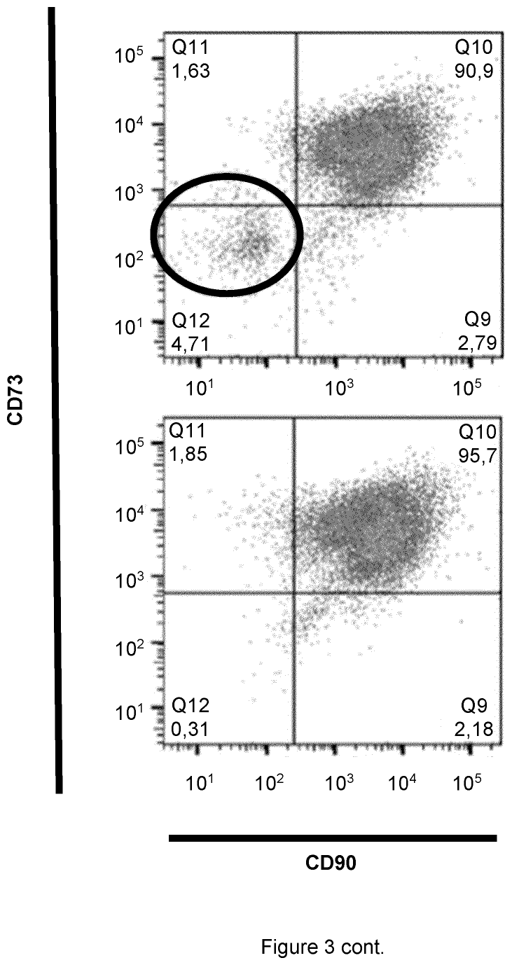

Cells were surface stained with fluorochrome-conjugated monoclonal antibodies against CD45, CD31, HLA class II DR, DP, DQ, CD73 and CD90 and a live/dead cell marker (DCM). Control cells containing a mixture of fetal MSC spiked with 4% of peripheral blood mononuclear cells (PBMC), were used to demonstrate the possibility to detect cells present at a concentration of: 5% which is the set acceptance limit for each tested contaminating cell type in the samples. FIG. 3 shows a typical flow cytometry plot (batch Q2/DP4).

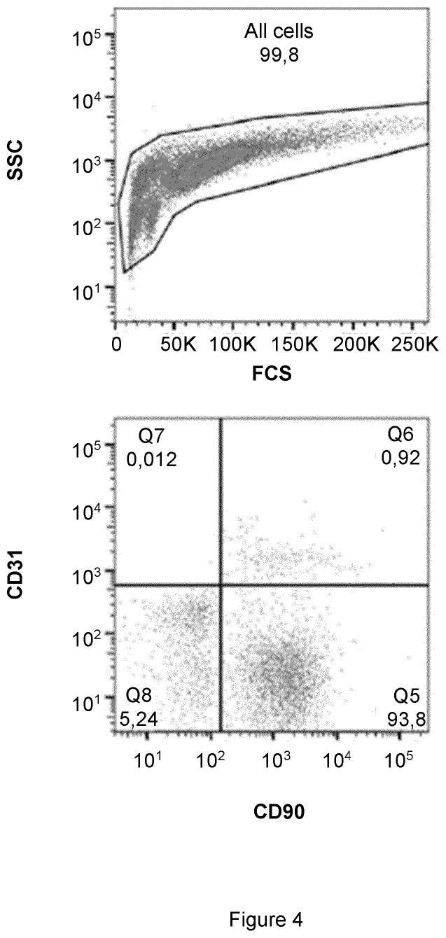

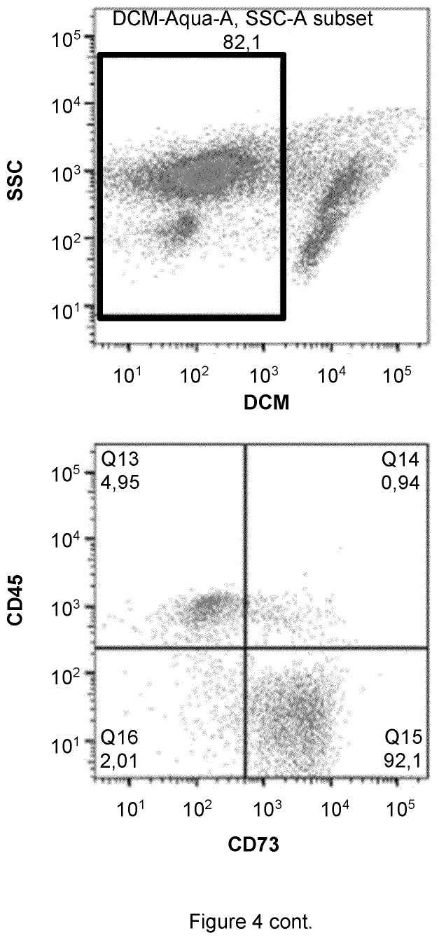

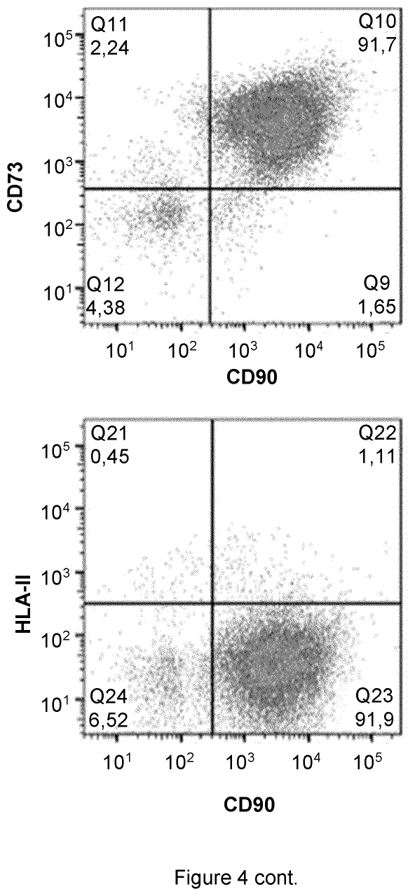

All stainings for analysis by flow cytometry were performed according to the following protocol: The cells were washed once with PBS and incubated with appropriate amounts of antibody at 4° C. for 30 min. Data acquisition was done on a FACS Calibur (BD) and data were analysed with FlowJo (TreeStar Inc.) software. A DCM was used for gating out dead cells but is not part of determination of viable cells in the FMSC drug product (viability is measured by Eosin exclusion). Gating was done on the fetal MSC control cells spiked with 4% PBMC by firstly in the FSC-SSC view setting a large gate on all cells (fetal MSC and PBMC). Then dead cells were excluded by gating on SSC vs DCM. Then CD90 is plotted against CD73 and quadrants are set to separate the positive and the negative populations. CD90 is then plotted against CD31, CD 45 and HLA-II, respectively and all gates are set. The set gates are then used for the un-spiked cells to determine the amount of cells positive for the respective marker. The control cells are fetal MSC that have been extensively analysed within the method, i.e. inter- and intra assay variability. The gating strategy is demonstrated in FIG. 4.

### Bone Differentiation Assay

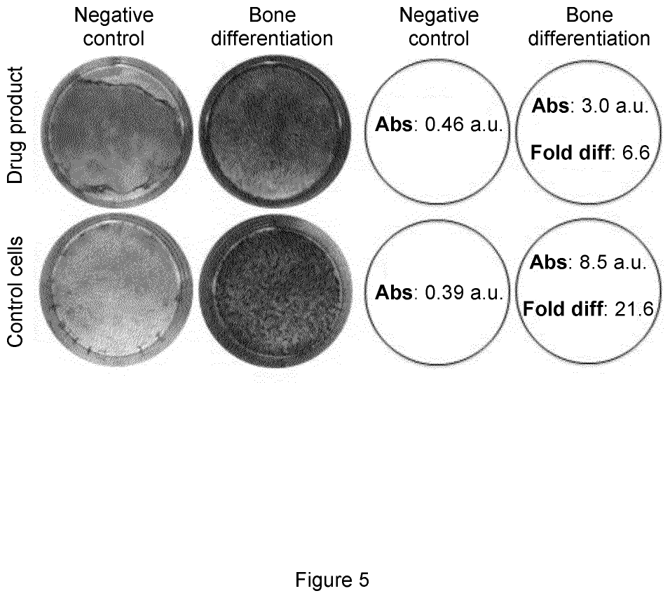

The ability of the fetal MSC to differentiate into osteoblasts is determined using an in vitro bone differentiation assay. Briefly, cryopreserved FMSC drug product and control cells are thawed and diluted to a cell concentration of 1.2×10⁴ viable cells/mL in the same medium used during production. One mL of cell suspension/well is seeded in hexaplicates. The control cells are fetal MSC that have been extensively analysed within the method, i.e. inter- and intra assay variability. After 24 hours of incubation at 37° C., the medium is changed, 3 wells with fresh culture medium, negative control, and 3 wells with StemMACS OsteoDiff medium, (Miltenyi) for differentiation. The medium is then replaced every 3-4 days until end of culture. After 14-21 days of culture depending on cell confluency, the cells are washed with PBS and 4% formalin is added for 1 hour to fix the cells. The cells are then washed with dH2O and stained with 1 mL freshly made 40 mM Alizarin Red S, pH 4.2 per well for 10 minutes at RT with rotation of the plate. Plates are then washed 5 times with dH2O and once with PBS and air dried. Photos are taken with 10× magnification. The staining is quantified by elution of the dye with 1 mL of 10% Cetylpyridinum chloride (CPC) and the optical density of the solution is measured with a Spectrophotometer at 562 nm using 10% CPC as a blank. Mean of the triplicate samples are calculated and results are given as fold differentiation over negative control. When the sample cells have a 2.5-fold differentiation over negative control, bone formation is considered to be positive. Representative data are presented in FIG. 5.

### Determination of Purity and Identity of FMSC Drug Product

A qualification of the flow cytometry method for the determination of the percentage of fetal MSC (CD73+CD90+) has been performed to ensure the acceptability of the performance of the analysis method and to perform a qualification (mainly ensuring the maintenance, good user routines and the suitability) of the flow cytometry instrument used for analysis.

A combination of markers that should be positive and negative are chosen to assess product purity and cellular impurities The positive markers are CD73 and CD90 and the negative markers are CD45, CD31 and HLA class II. The quantification was compared using two different flow cytometry instruments. A control sample consisting of an MSC sample spiked with approximately 4% viable PBMC was used to ensure the possibility to detect the negative markers since PBMC preparations contains cells expressing CD45 and CD31. The control sample is included in every assay for trending purposes to detect drifting of the assay.

HLA class II is analyzed in order to detect the presence of activated cells. As no activated cells are present in the control cells, the following is done to verify the ability to detect this marker: For every new batch of HLA class II antibody, or once per year, a control sample of activated MSC are added to the analysis.

### Potency: Bone Forming Capacity

The bone differentiation assay is the functional assay for the FMSC drug product. The purpose of the qualification was to determine lower limit of detection, precision and intermediate precision. Fetal MSC from four different donors, one of them expanded into 3 different FMSC drug product-batches, were used in the qualification: MSC1, MSC24, MSC26, Q2/DP2, Q2/DP3, Q2/DP4. The number of days to run the assay was specified by testing MSC1 with readings of independent triplicates after 14, 16 and 20 days. Data showed that samples should be cultured for >14 days to obtain a positive read-out of >2.5-fold differentiation over negative control (=[the mean absorbance of the differentiated cells in triplicate]/[the mean absorbance of the undifferentiated cells in triplicate] should be >3). The same sample was also analysed at three other independent occasions (mean fold difference 8.4, Std dev 2 and 25% CV), further verifying that this cultivation time is suitable for a good read-out. Prolonged culture can lead to lost cells due to overcrowding of the culture area.

### REFERENCES

Ramesh et al., Trophic effects of multiple administration of mesenchymal stem cells in children with osteogenesis imperfecta. Clin. Transl. Med. 2021; 11:e385.

### Items

1. A population of FMSCs derived from fetal liver, wherein the FMSCs
a) have the ability to expand;
b) are non-tumorigenic;
c) are adherent without GF;
d) are CD73+, CD90+, CD45−, CD31−, and HLA class II−; and
e) can differentiate into osteoblasts.

2. The population of FMSCs according to item 1, wherein the fetal liver is isolated from elective abortion in first trimester.

3. The population of FMSCs according to any one of the preceding items, wherein the FMSCs are highly bone forming.

4. The population of FMSCs according to any one of the preceding items, wherein the differentiation of the FMSCs into bone is at least two-fold differentiation over negative control.

5. The population of FMSCs according to any one of the preceding items, wherein the FMSCs can expand and grow for 13 passages.

6. The population of FMSCs according to any one of the preceding items, wherein the FMSCs have a Hayflick limit.

7. The population of FMSCs according to any one of the preceding items, wherein the population of FMSCs is at least 85% CD73+.

8. The population of FMSCs according to any one of the preceding items, wherein the population of FMSCs is at least 85% CD90+.

9. The population of FMSCs according to any one of the preceding items, wherein the population of FMSCs is at least 70% are live cells.

10. The population of FMSCs according to any one of the preceding items, wherein the population of FMSCs is no more than 5% CD45+.

11. The population of FMSCs according to any one of the preceding items, wherein the population of FMSCs is no more than 5% CD31+.

12. The population of FMSCs according to any one of the preceding items, wherein the population of FMSCs is no more than 5% HLA class II+.

13. The population of FMSCs according to any one of the preceding items, wherein the population of FMSCs is suitable for allogeneic transplantation.

14. A population of FMSCs comprising at least 70% viable cells expressing at least 85% CD73, and at least 85% CD90, and expressing no more than 5% of CD45, CD31 and HLA class II.

15. A method for expanding a population of FMSCs comprising culturing said cells in adherent cell culture in cell culture medium with no growth factors.

16. A method for enriching a primary fetal liver explant for FMSCs comprising
a) isolation of fetal MSCs from the dissected fetal liver tissue;
b) expansion in adherent culture in cell culture medium with no growth factors;
c) passaging the cells; and
d) harvesting the cells.

17. The method according to item 16, wherein passaging the cells comprises removing dead cells and/or non-MSC by washing such that only adherent cells remain in the culture.

18. The method according to item 17, wherein the cells are passaged at 70-100% confluency.

19. A method for release testing of a population of fetal mesenchymal stem cells (FMSCs) derived from fetal liver for use in treatment of osteogenesis imperfecta, said method comprising determining in the population of FMSCs
a) expression of CD73 and CD90; and
b) absence or low expression of CD45, CD31 and HLA class II.

20. The method according to item 19, wherein the population of FMSCs is determined to have at least 70% viable cells expressing at least 85% CD73, and at least 85% CD90, and no more than 5% of CD45, CD31 and HLA class II.

21. The method according to any one of items 19 to 20, wherein the release testing comprises immunophenotyping.

22. The method according to item 21, wherein the immunophenotyping is done by flow cytometry.

23. The method according to item 21, wherein the immunophenotyping is done by immunocytochemistry.

24. The method according to any one of items 19 to 23, wherein the population is further determined to be able to expand, being non-tumorigenic, and being able to differentiate into osteoblasts.

25. The method according to any one of items 19 to 24, wherein the population is further characterized as being one or more of sterile, virus free, mycoplasma free, exhibiting chromosome stability, being free of known mutations causing skeletal dysplasia, and being a colourless cell suspension free of visible particulate matter.

26. A population of FMSCs according to any one of items 1 to 14 for use in the treatment of osteogenesis imperfecta.

27. The population of FMSCs for use according to items 26, wherein the population of FMSCs is administered intravenously.

28. The population of FMSCs for use according to items 26, wherein the population of FMSCs is administered postnatally and/or prenatally.

29. The population of FMSCs for use according to items 26, wherein the population of FMSCs is administered four times postnatally.

30. The population of FMSCs for use according to items 26, wherein the population of FMSCs is administered one time prenatally and three times postnatally.

31. The population of FMSCs for use according to items 26 to 30, wherein the population of FMSCs is suitable for allogenic transplantation.

1. A population of fetal mesenchymal stem cells (FMSCs) derived from fetal liver, wherein the FMSCs
a) have the ability to expand;
b) are non-tumorigenic;
c) are adherent without growth factors (GF);
d) are CD73+, CD90+, CD45−, CD31−, and HLA class II−; and
e) can differentiate into osteoblasts.

2. The population of FMSCs according to any one of the preceding claims, wherein the FMSCs are bone forming, produce healthy collagen and secrete paracrine factors.

3. The population of FMSCs according to anyone of the preceding claims, wherein the differentiation of the FMSCs into bone is at least two-fold differentiation over negative control.

4. The population of FMSCs according to any one of the preceding claims, wherein the FMSCs can expand and grow for 13 passages.

5. The population of FMSCs according to any one of the preceding claims, wherein the FMSCs have a Hayflick limit.

6. The population of FMSCs according to any one of the preceding claims, wherein the population of FMSCs is at least 85% CD73+.

7. The population of FMSCs according to any one of the preceding claims, wherein the population of FMSCs is at least 85% CD90+.

8. The population of FMSCs according to any one of the preceding claims, wherein the population of FMSCs is at least 70% live cells, such as at least 85% are live cells.

9. The population of FMSCs according to any one of the preceding claims, wherein the population of FMSCs is no more than 5% CD45+.

10. The population of FMSCs according to any one of the preceding claims, wherein the population of FMSCs is no more than 5% CD31+.

11. The population of FMSCs according to any one of the preceding claims, wherein the population of FMSCs is no more than 5% HLA class II+.

12. The population of FMSCs according to any one of the preceding claims, wherein the population of FMSCs is suitable for allogeneic transplantation.

13. The population of FMSCs according to any one of the preceding claims, comprising at least 85% viable cells.

14. A population of FMSCs according to any one of claims 1 to 13 for use in the treatment of osteogenesis imperfecta.

15. The population of FMSCs for use according to claim 14, wherein the population of FMSCs is administered postnatally and/or prenatally.

16. A method for release testing of a population of fetal mesenchymal stem cells (FMSCs) derived from fetal liver for use in treatment of osteogenesis imperfecta, said method comprising determining in the population of FMSCs
a) expression of CD73 and CD90; and
b) absence or low expression of CD45, CD31 and HLA class II.

17. The method according to claim 16, wherein the population of FMSCs is determined to have at least 70% viable cells expressing at least 85% CD73, and at least 85% CD90, and no more than 5% of CD45, CD31 and HLA class II.

18. The method according to any one of claim 16 to 17, wherein the release testing comprises immunophenotyping.

19. The method according to claim 18, wherein the immunophenotyping is done by flow cytometry.

20. The method according to claim 18, wherein the immunophenotyping is done by immunocytochemistry.

21. The method according to any one of claims 16 to 20, wherein the population is further determined to be able to expand, being non-tumorigenic, and being able to differentiate into osteoblasts.

22. The method according to any one of claims 16 to 21, wherein the population is further characterized as being one or more of sterile, virus free, mycoplasma free, exhibiting chromosome stability, being free of known mutations causing skeletal dysplasia, and being a colourless cell suspension free of visible particulate matter.
